(19) United States
(12) Patent Application Publication
(10) Pub. No.: US 2025/0304901 A1
(43) Pub. Date: Oct. 2, 2025

(54) PNEUMATIC TRANSFER OF FLUID SAMPLES OVER LENGTHY CONDUITS

(71) Applicant: Waters Technologies Corporation, Milford, MA (US)

(72) Inventors: Sylvain G. Cormier, Mendon, MA (US); Christian Zeigler, Milford, MA (US)

(21) Appl. No.: 19/091,493

(22) Filed: Mar. 26, 2025

Related U.S. Application Data
(60) Provisional application No. 63/569,970, filed on Mar. 26, 2024.

(51) Int. Cl. C12M 1/00; G01N 35/10

(52) U.S. Cl. CPC C12M 29/00; G01N 35/10

(57) ABSTRACT

Exemplary embodiments may provide a pneumatic transfer system for transferring fluid samples from a bioreactor to sample processing system without the need for manual transfer of the fluid samples. The exemplary embodiments may transfer fluid samples distances of at least 1 foot with the pneumatic transfer system without substantial loss of the fluid samples. Exemplary embodiments may recover at least 50% of the mass of the fluid samples at the destination. Exemplary embodiments may choose the pressure applied by one or more pumps so as to be sufficient enough so as to move the fluid samples at a reasonable rate without causing fragmentation of the fluid samples that results in non-negligible portions of the fluid samples sticking to the inner walls of the conduit in small droplets.

### CROSS-REFERENCE TO RELATED APPLICATIONS

This application claims the benefit of and priority to U.S. Provisional Patent Application No. 63/569,970, filed Mar. 26, 2024, the entire contents of which are hereby incorporated by reference.

### BACKGROUND

Bioreactors carry out chemical processes involving organisms or biochemical active substances derived from organisms. Examples of bioreactors include bioreactors for bioconversion of corn to ethanol, bioreactors for sewage treatment, bioreactor for creating drugs, bioreactors for inactivating cells, etc. In the course of operation of bioreactors, one may wish to obtain fluid samples from the bioreactors and process those fluid samples. Conventionally these samples are manually carried to sample preparation equipment that prepare the samples for processing, such as analysis. The sample preparation equipment typically is located a distance in excess of 20 feet from the bioreactors, such as in a separate room.

### SUMMARY

In accordance with a first inventive facet, a pneumatic fluid transport system includes a conduit for carrying a fluid from a source to a destination. The transport of the fluid is automated so that there is no need for manual transport of the sample. The conduit may be at least 1 foot in length. The pneumatic fluid transport system may also include one or more pumps or vacuum sources secured to the conduit for applying a force to a gas for transporting the fluid along the length of the conduit. The one or more pumps or vacuum sources may be configured to move the fluid at a rate such that at least 50% of the mass of the fluid is recovered at the destination.

The conduit may include one or more tubes. One end of the conduit may be secured to a bioreactor from which the fluid is extracted. Another end of the conduit may be secured to sample processing equipment for processing a sample of the fluid. The one or more pumps or vacuum sources may move the fluid at a flow rate of between 1 mL/min and 6 mL/min while transporting the fluid along the length of the conduit. The conduit may have an inner diameter in the range of 0.3125 inches to 0.1 inches. The one or more pumps may be secured to the conduit for applying the force to the gas for transporting the fluid, and the one or more pumps may include a peristaltic pump. The conduit may be at least 40 feet in length. At least 50% of the mass of the fluid may be recovered at the destination. The fluid may be a volume of 10 μL to 5 mL.

In accordance with another inventive facet, a system includes a bioreactor for providing a sample in fluid form and a conduit connected to the bioreactor for receiving the sample. The conduit may be at least 1 foot in length. The system may further include a first pump connected to the tube for creating a pressure relative to the bioreactor to transport the sample in fluid form along the conduit. The first pump may be configured to move the fluid with the pressure at a flow rate of between 1 mL/min and 6 mL/min. The system additionally may include a first valve having a first position for receiving the sample into a sample loop and a second position to dispensing the sample out of the sample loop to a fluid path leading to sample processing equipment.

The first valve may include a port connected to a fluid path leading to a second pump and a second valve. The second valve may have a position for connecting with a wash source so that wash solution may be drawn and passed by the second pump to waste. The second valve may have a position for connecting with an air source so that air may be drawn and passed by the second pump through the sample loop. The conduit may have an inner diameter in the range of 0.3125 inches to 0.1 inches.

In accordance with an additional inventive facet, a method includes extracting a liquid sample from a bioreactor and applying a pressure to a conduit that contains the liquid sample having an initial mass to transport the liquid sample at least 1 foot along the conduit at a flow rate between 1 mL/min and 6 mL/min so as to recover at least 50% of the initial mass of the liquid sample after transporting the liquid sample at least 1 foot.

The conduit may have an inner diameter in the range of 0.3125 inches to 0.1 inches. The conduit may be hydrophobic. The liquid sample may be transported at least 40 feet. The fluid sample may contain at most 20 packets of fluid.

### BRIEF DESCRIPTION OF DRAWINGS

FIG. 1A depicts a draw phase of an illustrative pneumatic fluid transfer system of an exemplary embodiment.

FIG. 1B depicts a dispense phase of an illustrative pneumatic fluid transfer system of an exemplary embodiment.

FIG. 1C depicts a weak wash phase of an illustrative pneumatic fluid transfer system of an exemplary embodiment.

FIG. 1D depicts a strong wash phase of an illustrative pneumatic fluid transfer system of an exemplary embodiment.

FIG. 1E depicts an air flush phase of an illustrative pneumatic fluid transfer system of an exemplary embodiment.

FIG. 2A depicts a draw phase of an alternative illustrative pneumatic fluid transfer system of an exemplary embodiment.

FIG. 2B depicts a dispense old sample phase of the alternative illustrative pneumatic fluid transfer system of an exemplary embodiment.

FIG. 2C depicts a first dispense sample phase of the alternative illustrative pneumatic fluid transfer system of an exemplary embodiment.

FIG. 2D depicts a draw strong wash phase of the alternative illustrative pneumatic fluid transfer system of an exemplary embodiment.

FIG. 2E depicts a draw air phase of the alternative illustrative pneumatic fluid transfer system of an exemplary embodiment.

FIG. 2F depicts a draw weak wash phase of the alternative illustrative pneumatic fluid transfer system of an exemplary embodiment.

FIG. 2G depicts a dispense wash phase of an alternative illustrative pneumatic fluid transfer system of an exemplary embodiment.

FIG. 3A depicts a block diagram of components of an illustrative pump of exemplary embodiments.

FIG. 3B depicts a block diagram of components of a controller of the pump of the exemplary embodiments.

FIG. 4 depicts a flowchart of steps for constructing, configuring, and using the pneumatic fluid transfer systems of exemplary embodiments.

### DETAILED DESCRIPTION

Exemplary embodiments may provide a pneumatic fluid transport system for transferring fluid samples from a bioreactor to a sample processing system without the need for manual transfer of the fluid samples. The exemplary embodiments may transfer fluid samples distances of at least 1 foot with the pneumatic fluid transport system without substantial loss of the fluid samples. Exemplary embodiments may recover at least 50% of the mass of the fluid samples at the destination (e.g., at the sample preparation equipment or end of the conduit over which the fluid samples are transported).

The pneumatic fluid transport system of the exemplary embodiments may use one or more pumps, such as peristaltic pumps, to create pressure to move the fluid samples over a conduit. In some exemplary embodiments, the one or more pumps create pressure in the conduit to pull the fluid samples along the conduit from starting point to destination. In other exemplary embodiments, the one or more pumps may generate air pressure to push the fluid samples along the conduit. Exemplary embodiments may choose the air pressure applied by the one or more pumps so as to be sufficient enough so as to move the fluid samples at a reasonable rate without causing fragmentation of the fluid samples that results in non-negligible portions of the fluid samples sticking to the inner walls of the conduit in small droplets.

In addition, the affinity of the conduit is chosen to be the opposite of the affinity of the fluid sample in exemplary embodiments. For instance, a hydrophobic conduit may be chosen where the fluid samples are hydrophilic. Where the fluid samples are hydrophilic, the conduit may be chosen to hydrophobic. By choosing such opposite affinities, exemplary embodiments may reduce the tendency of the fluid samples to stick to the inner walls of the conduit and may ultimately reduce the percentage of mass of the liquid samples that is not recovered at the destination.

A “fluid’ refers to a substance that has no fixed shape and that is not a solid. A fluid encompasses a gas, a liquid or combinations thereof. In addition, a fluid may include a liquid containing matter such as cells and the like.

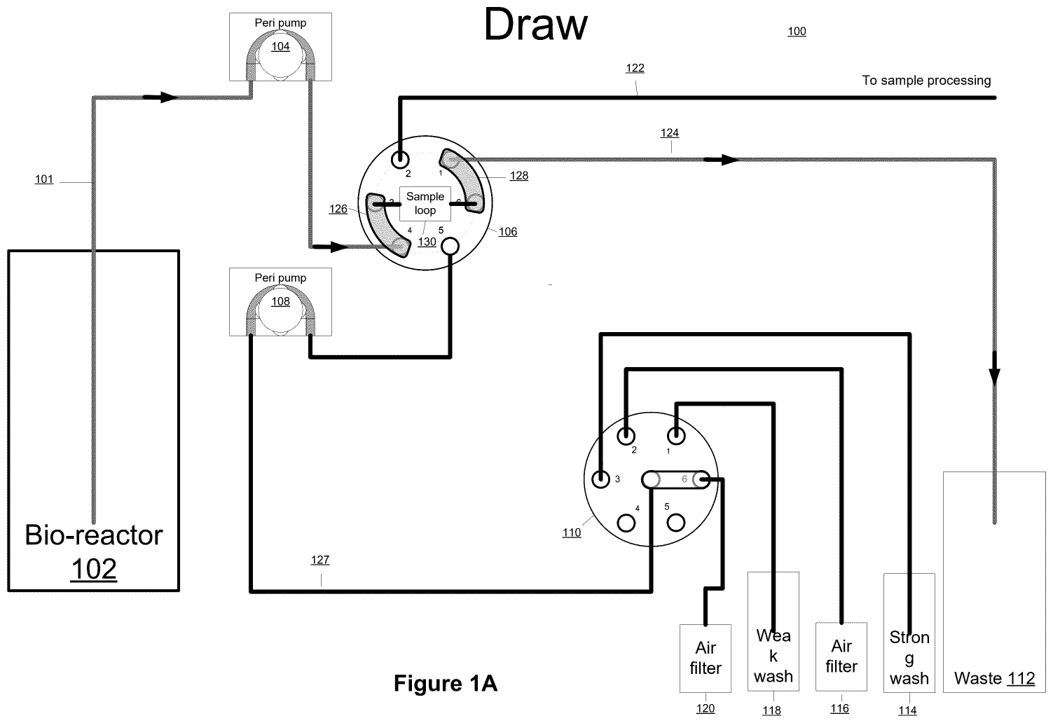

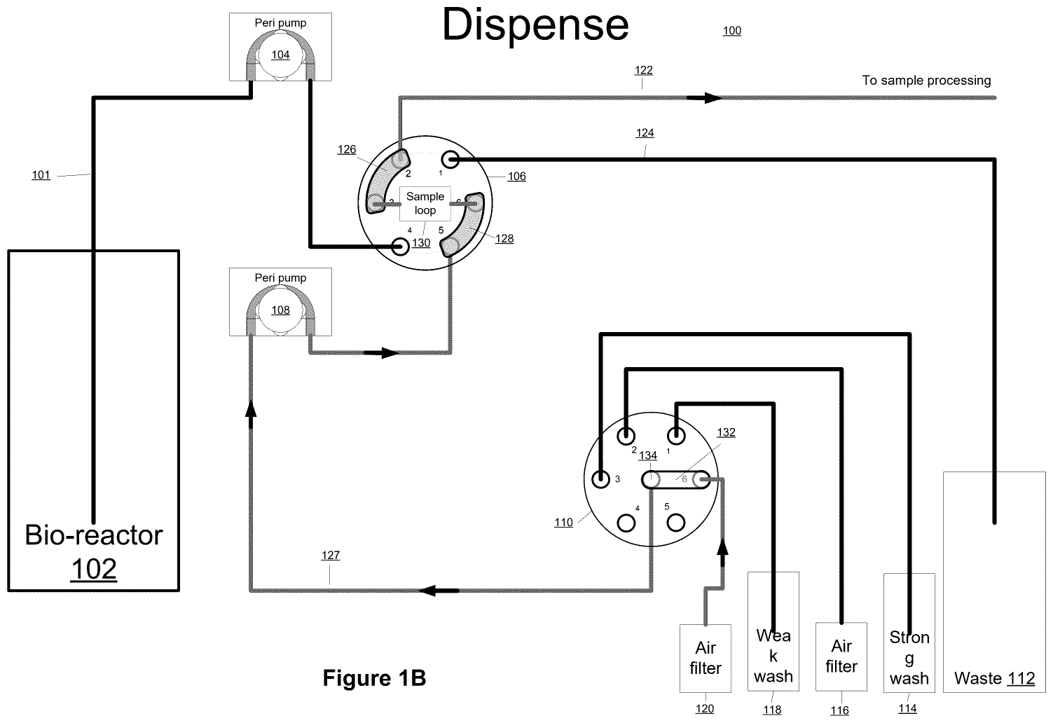

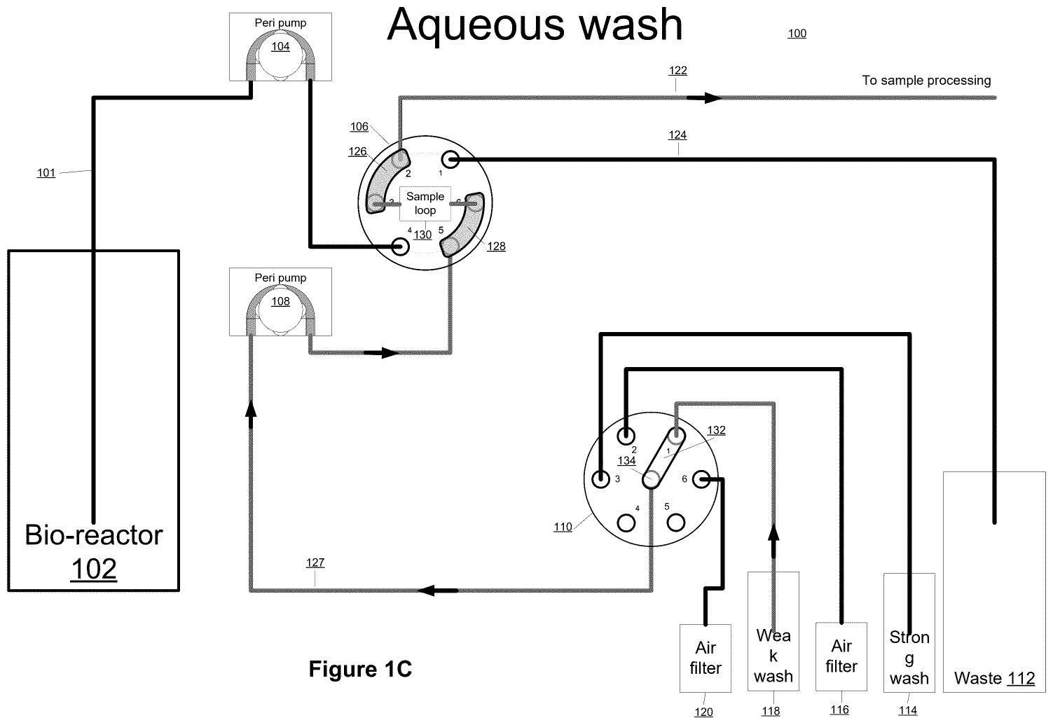

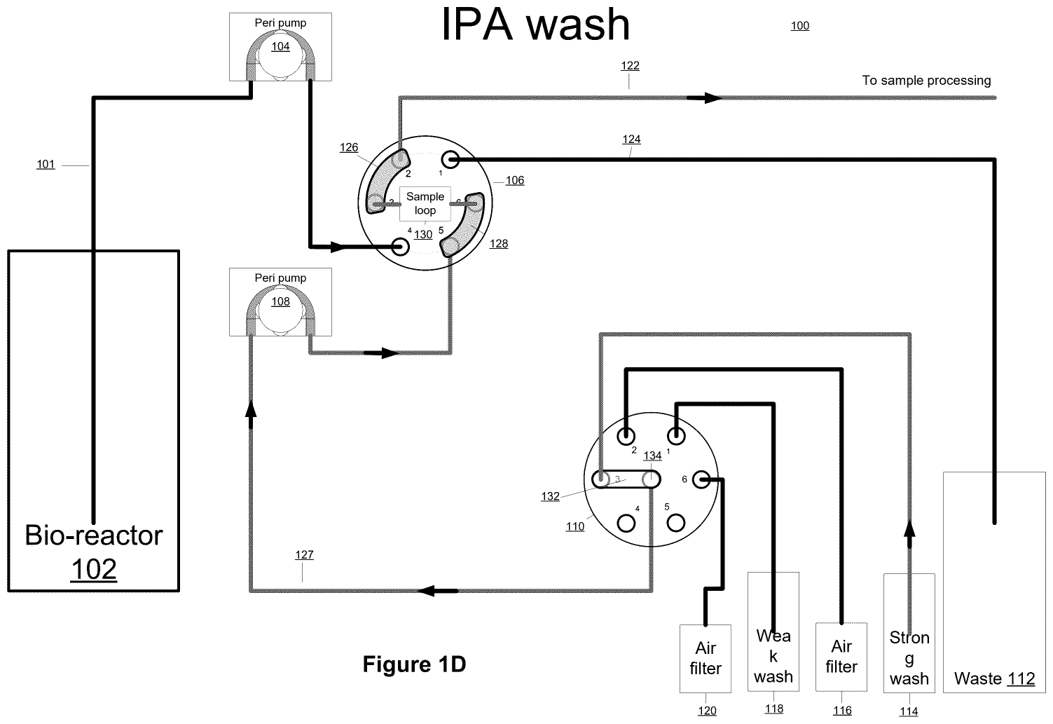

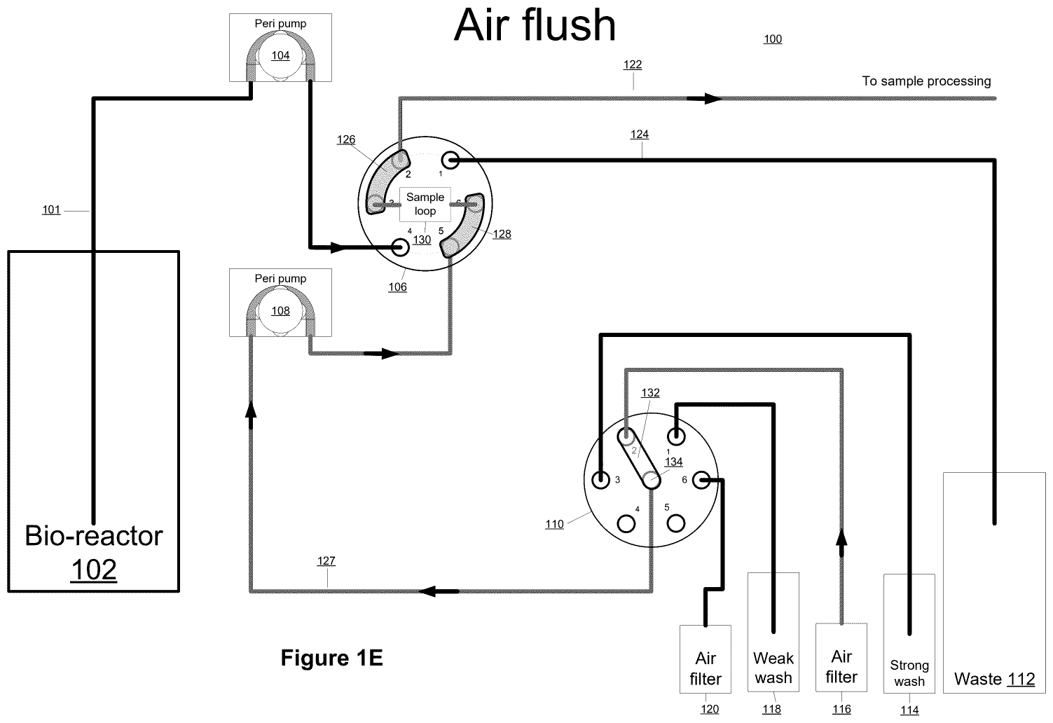

FIGS. 1A-1E depict different phases of operation of an illustrative pneumatic fluid transport system 100 in accordance with exemplary embodiments. The phases include a draw phase, a dispense phase, a weak wash phase, a strong wash phase, and an air flush phase. These phases and the elements of the pneumatic fluid transfer system are described below.

FIG. 1A depicts an illustrative pneumatic fluid transport system 100 of an exemplary embodiment. In this pneumatic fluid transport system 100, the fluid samples may be pulled from a bioreactor 102 rather than pushed from the bioreactor 102. A sampling mechanism may be used to gather fluid samples from the bioreactor 102. The bioreactor 102 serves as a source of the fluid samples. The samples may comprise liquid containing cells and/or other organic and inorganic matter. The fluid samples are extracted from the bioreactor 102 and pulled along a conduit 101 by a suction force created by pump 104. The conduit 101 may be, for example, a glass or plastic tube that is connected to the pump 104. The pump 104 may be a peristaltic pump or another variety of suitable pump for creating the negative pressure relative to the bioreactor 102. As will be described in more detail below, the pump 104 should be configured to exert a sufficient force so as to move the fluid samples at a desired rate without causing evaporation of the fluid samples and without causing the fluid samples to fragment excessively. The conduit 101 extends to a valve 106.

The valve 106 may be, for example, a rotary disk valve that has a rotor and a stator. In the valve 106 that is depicted there are six ports (numbered 1-6). Traces 126 and 128 are provided in the rotor. In the draw phase shown in FIG. 1A, a sample is drawn from the bioreactor and held in a sample loop 130. As shown, the trace 126, connects ports 3 and 4, and trace 128 connects ports 6 and 1. As such, the fluid sample enters port 4 and is pushed over trace 126 to a sample loop 130. The output of the sample loop 130 is connected to port 6. Port 6 is connected to port 1, which, in turn, is connected to conduit 124 that leads to waste 112. In this position of the valve 106, the fluid samples are drawn from the bioreactor 102 and at least a portion of the sample is captured in the sample loop 130.

FIG. 1B depicts the dispense phase for valve 106 for dispensing the fluid sample held in the sample loop 130 to the sample processing equipment via conduit 122. Conduit 122, like conduit 101, may be, for example, a glass or plastic tube. In FIG. 1B, the valve 106 is in a different position so that trace 126 connects ports 2 and 3, and trace 128 connects ports 5 and 6. In this position, air from an air source passes through air filter 120 into port 6 of valve 110. The air flows through trace 132 to a central port 134. The central port 134 is connected to a conduit 127. A pump 108 creates pressure to draw the air via conduit 127 to port 5 of the valve 106. Pump 108 may be, for instance, a peristaltic pump. The air enters port 5, travels through trace 128 to port 6. The air at port 6 pushes the fluid sample out of the sample loop 130 to port 3. The sample is pushed over trace 126 to port 2. Port 2 is connected to conduit 122 that leads to the sample processing equipment. For example, the sample processing equipment may include but is not limited to a storage, an instrument, another reactor, or even the original bioreactor. Hence, the sample exits port 2 and travels over conduit 122 to the sample processing equipment.

After the fluid sample is dispensed, it may be desirable to wash the sample loop 130 and the fluid path over which the sample was transported. This may be achieved in part by the weak wash phase depicted in FIG. 1C. The traces 126 and 128 of valve 106 remain in the same positions as they were in the dispense position of FIG. 1B. However, the rotor of valve 110 is rotated so that trace 132 connects port 1 with the central port 134. Port 1 is connected to a source of weak aqueous wash 118. The weak aqueous wash passes along conduit 127 to port 5 of valve 106 and through the sample loop 130. The weak wash then passes out port 2 to conduit 122 to the sample processing equipment.

A strong wash also may be applied. The strong wash phase depicted in FIG. 1D is like that of the weak wash phase of FIG. 1C except that the trace 132 connects the central port 134 with port 3. Port 3 is connected to a source of strong wash 114. The strong wash passes through conduit 127, the sample loop 130, and conduit 122 to the sample processing equipment.

FIG. 1E depicts the valve positions for the air flush phase, which flushes air through the sample loop and the conduit 122. The valve 106 stays in a position like that of the strong wash of FIG. 1D. However, valve 110 is in a different position than in FIG. 1D. The rotor of valve 110 is rotated so that trace 132 interconnects port 2 and the central port 134. Port 2 is connected to an air source with an air filter 116. As a result, air from the air source 116 flushes out the sample loop 130 and the conduit 122.

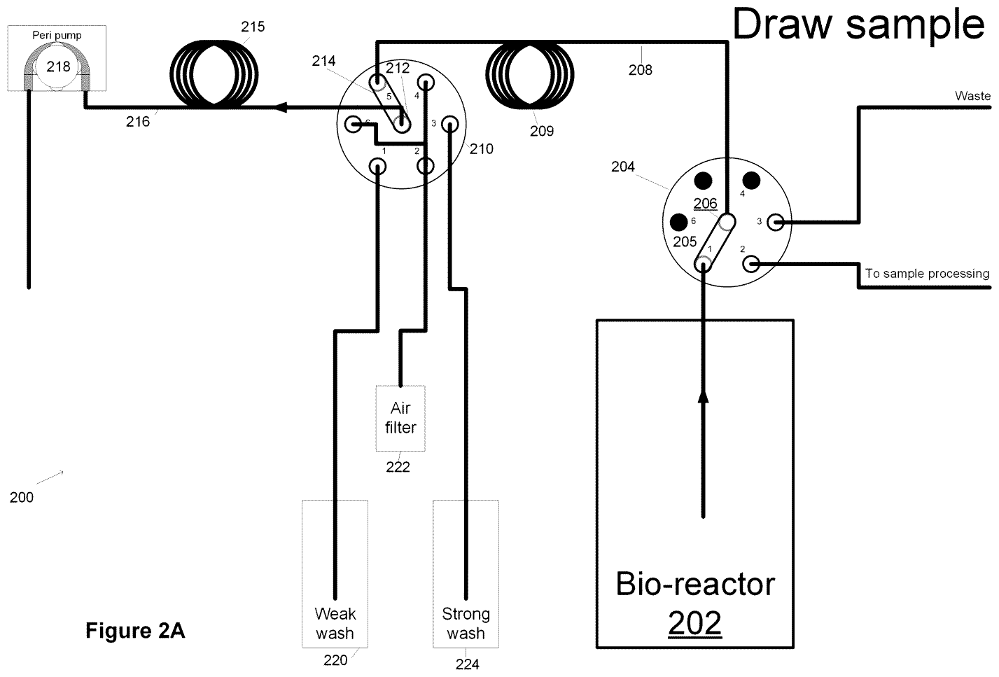

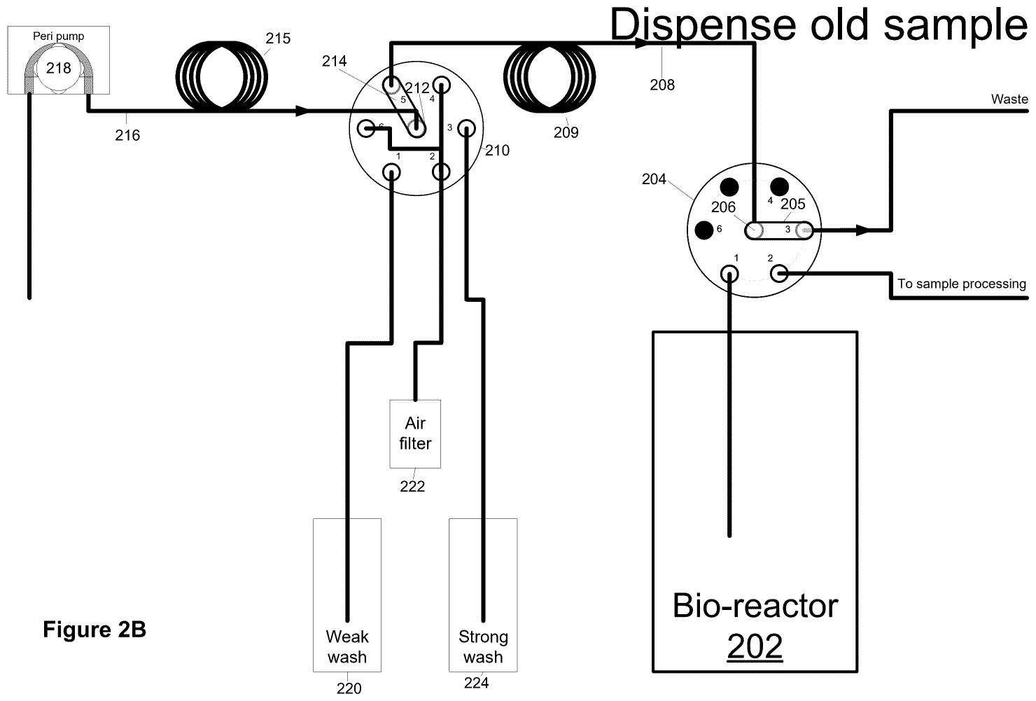

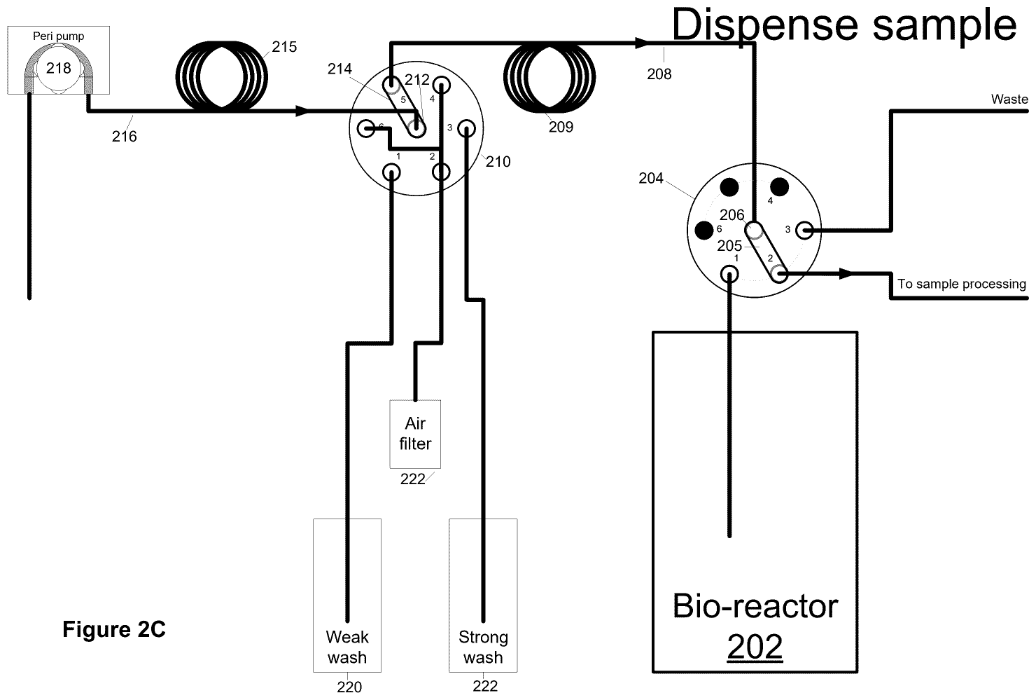

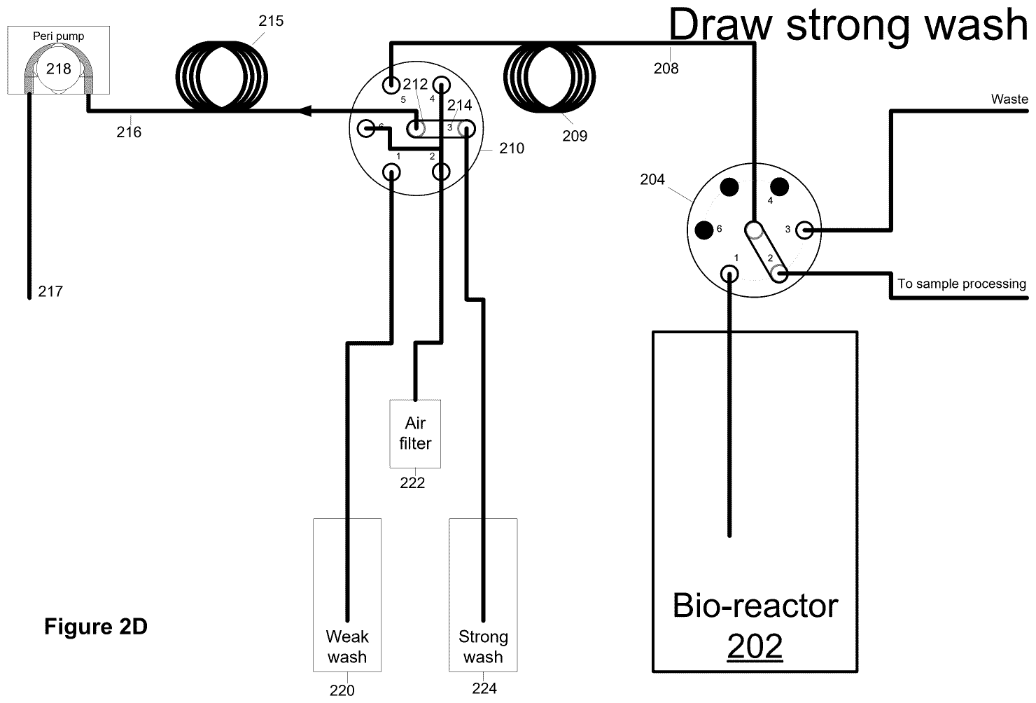

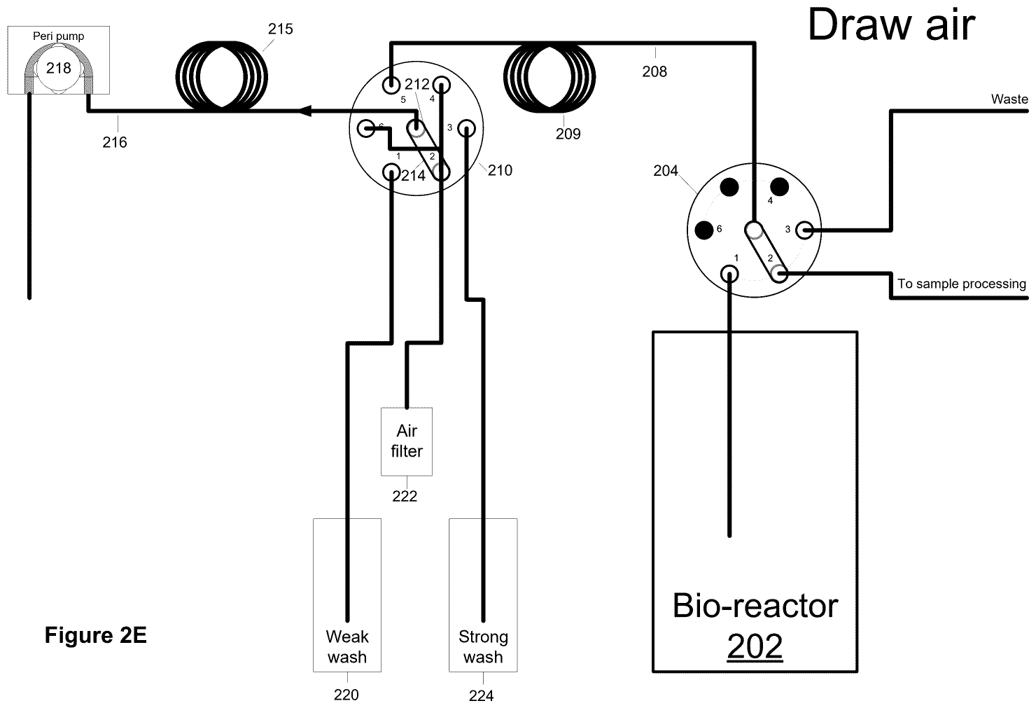

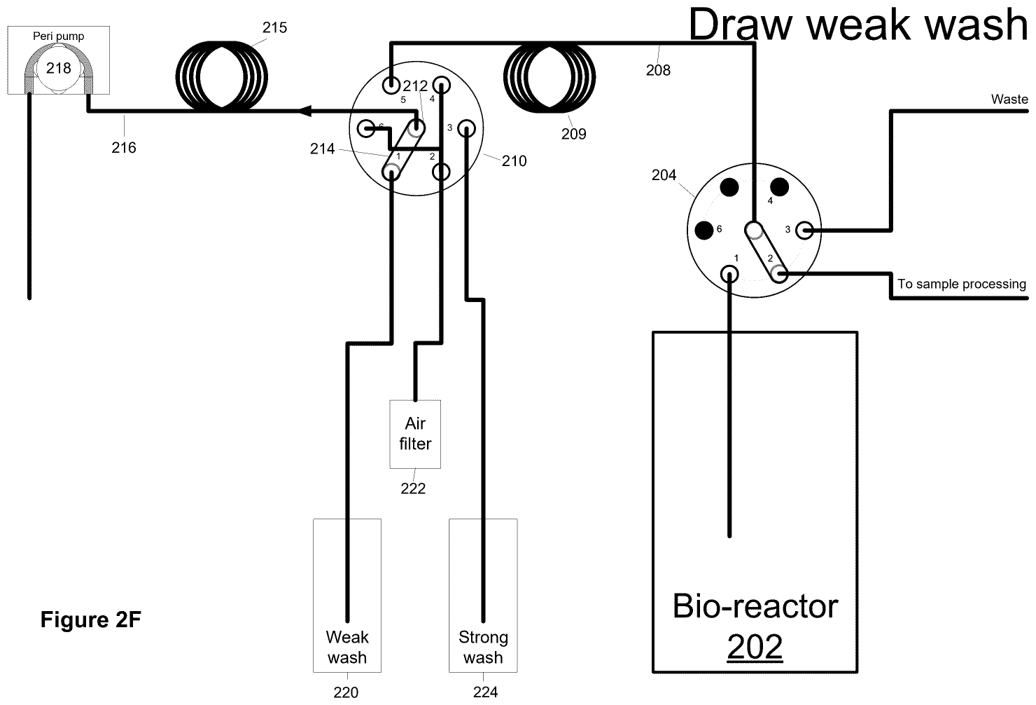

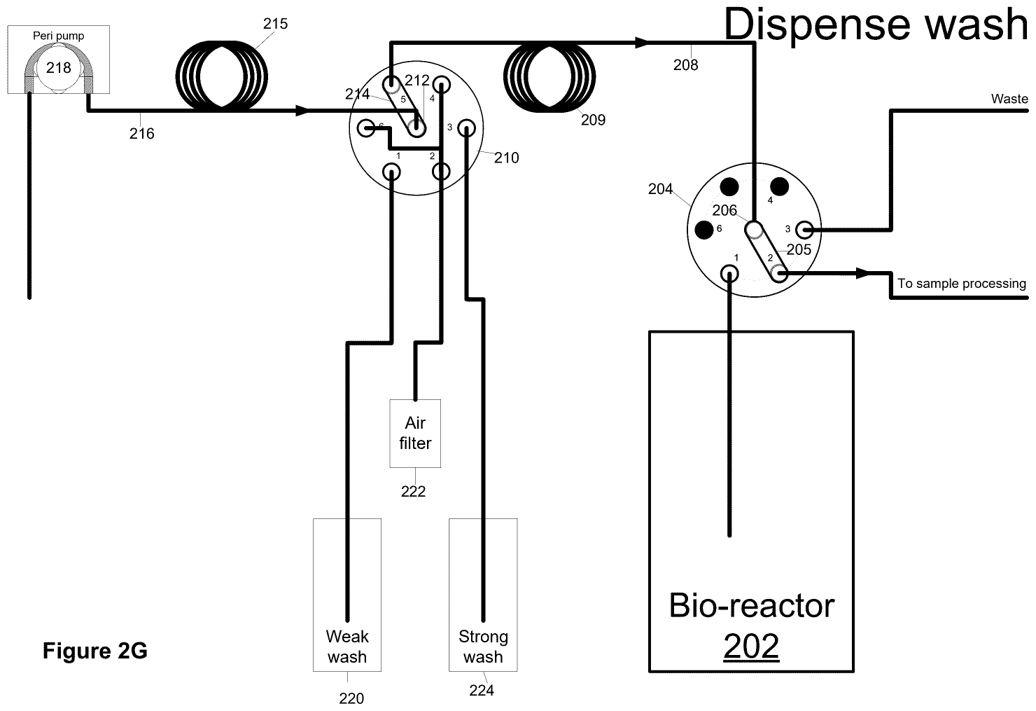

FIGS. 2A-2G depict an alternative pneumatic fluid transport system 200 in accordance with exemplary embodiments. The general flow in this alternative system 200 is to draw a sample, push the sample to waste, draw another sample that usually will be overfilled, push the overfill to waste, and push the sample to the sample processing equipment. These stages are discussed below. Washing stages may also be included.

FIG. 2A depicts a draw sample phase of the alternative pneumatic fluid transport system 200. A pump 218 is used in this alternative pneumatic fluid transport system 200. The pump 218 may be a peristaltic pump or another variety of pump. The fluid sample may be drawn from the bioreactor 202 by the negative pressure created by the pump 218. The bioreactor 102 is connected to valve 204 via port 1. A trace 205 connects port 1 with central port 206. The central port 206 is connected to a conduit 208 that includes a sample loop 209. In some embodiments, the conduit 208 comprises the sample loop itself. The conduit 208 may be, for example, a glass or plastic tube. Thus, the fluid passes through port 1, trace 205, and central port 206 on to conduit 206. The conduit 208 may be connected to port 5 of valve 210. Trace 214 may connect port 5 with a central port 212 to sample loop 215 via conduit 216. Hence, the fluid samples may be loaded into the sample loop 209 and overflow and solvent may be loaded into sample loop 215 if necessary in this phase. The sample loop 215 may be realized as part of the conduit 208 in some instances.

FIG. 2B depicts the phase that dispenses the sample to waste. This is done to remove the sample from the bioreactor 202 to valve 204. The position of valve 204 changes. The valve can be rotated in either direction, clockwise or counter-clockwise, and chosen to rotate in a way that may or may not contact ports in order to prevent or allow the contact of those ports with any fluid that may be in the valve trace. In some embodiments, ports are connected fluidically to additional equipment. In some embodiments, ports are plugged. In some embodiments, there is no port present, for example, ports 4, 5, and 6 of valve 204 in FIG. 2 may not be present. In some embodiments, the elimination of ports reduces the potential for contamination since no unswept volume is present on the valves. In FIG. 2B, the rotor has been rotated so that trace 205 connects central port 206 with port 3 of the valve 204. A conduit leading to waste is connected to port 3. Hence, instead of the sample going to the sample processing equipment, the sample goes to waste. Subsequently, another sample is drawn to produce a fresh sample. Cleaning procedures can be performed between all of these steps. The cleaning steps are described in more detail below.

In some embodiments, an intermediate step may be performed when the sample loop 209 is overfilled, resulting in the filling of sample loop 215. The excess sample in sample loop 215 may be pushed to waste by connecting port 4 or 6 of valve 210 to a waste container. This helps for a fixed sample size to be obtained.

FIG. 2C depicts a dispense sample phase for dispensing the fluid sample of sample loop 209 to sample processing equipment. In this phase, pump 218 creates air pressure to push a fluid sample to the sample processing equipment. Conduit 216 is connected to the central port 212 of valve 210. The rotor has been rotated so that trace 214 connects the central port 212 with port 5 of the valve 210. Port 5 is connected to conduit 208. The air from pump 218 pushes the sample out of sample loop 209 to valve 204. Conduit 208 is connected to central port 206 of valve 204. The rotor of valve 204 has been rotated so that trace 205 connects the central port 206 with port 2 of the valve 204. Port 2 is connected to a conduit that leads to the sample processing equipment. This, the sample is transported out of the valve 204 to the sample processing equipment.

FIG. 2D depicts a cleaning phase where strong wash is drawn and run through the solvent loop 215 and conduit 216. Valve 210 is in a position where trace 214 connects port 3 with the central port 212. Port 3 is connected with a source of strong wash 224. The pump 218 applies a pressure so that the strong wash is drawn from the strong wash source 224 into port 3 of valve 210 and out the central port 212 to the conduit 216. The strong wash passes through the solvent loop 215 and conduit 216.

FIG. 2E depicts a phase where air is drawn and run through the solvent loop 215. Valve 210 is positioned so that trace 214 connects central port 212 with port 2. Port 2 is connected with an air source 222. In some embodiments, air source 222 comprises a filter. Pump 218 creates a suction force that draws air from the air source to valve 210 via port 2 and out central port 212 to conduit 216. The air passes into the sample loop 215 and conduit 216. In some embodiments, air may pass all the way through conduit 216 and pump 218. In some embodiments, pump 218 may instead draw air externally and push air through conduit 216 and sample loop 215.

FIG. 2F depicts a phase where a weak wash is draw into the solvent loop 215. Valve 210 is positioned so that trace 214 connects port 1 with central port 212. Port 1 is connected to a source of weak wash 220. The pump 218 applies a suction to draw the weak wash from the weak wash source to valve 210 via port 1. The weak wash is drawn out of the central port on to conduit 216 and through sample loop 214.

FIG. 2G depicts a phase where a wash or air is dispensed. In this phase, the pump 218 applies pressure to conduit 216 to push wash or air out of the conduit and sample loop 215. The wash is pushed to the central port 212 of the valve 210. Trace 214 connects the central port to port 5. Port 5 is connected to conduit 208. Hence, the wash or air is pushed our port 5 to conduit 208 and through sample loop 209. The wash or air is then pushed to central port 206 of valve 204. Since trace 205 connects the central port 206 with port 2, the wash or air is pushed out port 2 on to the sample processing equipment.

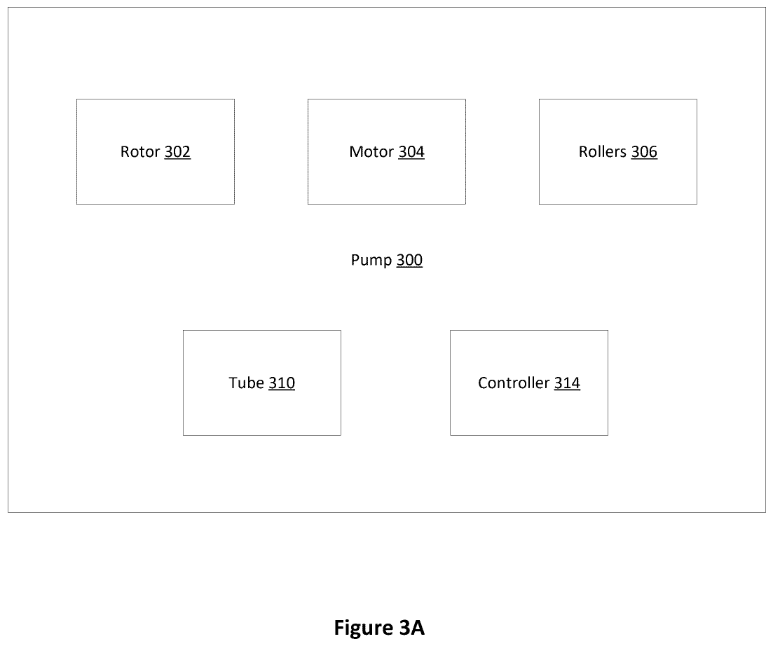

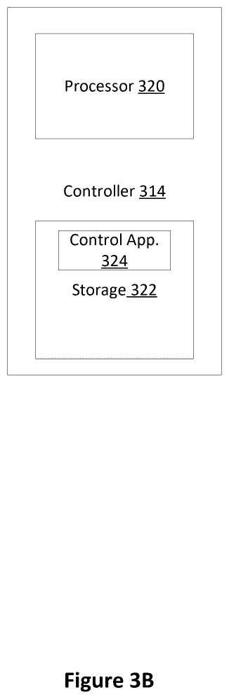

FIG. 3A depicts elements of an illustrative peristaltic pump 300 that may serve as a pump in the exemplary embodiments. The pump 300 may include a rotor 302 and a motor 304 for rotating the rotor 302. The pump may also include rollers 306. The pump 300 may include a flexible tube 310 through which fluid is to be displaced. During operation, the motor 304 causes the rotor 302 to rotate. The rollers 306 are secured to the outer circumference of the rotor 302 and act to pinch the tube 310 where they contact the tube. Fluid may be situated between two of the rollers 306 so as to urge it through the tube 310. The rollers 306 compress the tube as they rotate by and thus force the fluid to move through the tube 310. As the tube 310 becomes uncompressed and opens, more fluid is drawn into the tube 310. The operation of the pump 300 is under the control of the controller 314, which regulates the motor 304 to determine how quickly the rotor 302 rotates, which determines the magnitude of the positive air pressure or suction force produced by the pump 300.

FIG. 3B depicts a block diagram of illustrative components of the controller 314 in exemplary embodiments. The controller 314 may include a processor 320, such as a microprocessor, an application specific integrated circuit (ASIC), an field programmable gate array (FPGA), a central processing unit (CPU), a graphic processing unit (GPU) or the like. The controller 314 may include storage 322, such as random access memory (RAM), read only memory (ROM), solid state storage, optical storage, a hard disk, magnetic disk storage or combinations thereof. The storage 322 may hold computer programming instructions that are executable by the processor, such as a control application 324 for controlling operation of the pump 300 and the valves. It should be appreciated that the processor 320 may execute multiple control applications, libraries, methods, routines or the like in some exemplary embodiments. Moreover, the controller may be implemented via electrical circuitry rather than with a processor in some exemplary embodiments.

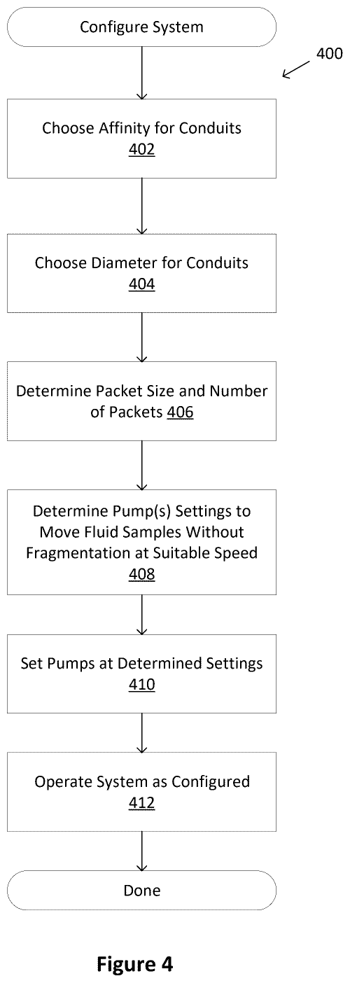

As was mentioned above, one challenge in moving fluids through a conduit in a pneumatic transfer system is fragmentation of the fluid samples. In order to avoid these issues, the pneumatic transfer system must be properly constructed and configured. FIG. 4 depicts a flowchart 400 of illustrative steps that may be performed in exemplary embodiments as part of the construction, configuration, and operation of the system.

One of the issues that may arise in a fluid transfer system such as the pneumatic fluid transport system of the exemplary embodiments is that there is an affinity mismatch. Having the fluid samples of a same affinity as the conduits of the pneumatic fluid transport system increases the likelihood of fragmentation during transport of fluid samples. Using a hydrophilic tube as a conduit with a hydrophilic fluid sample causes the sample to adhere more to the interior walls of the conduit. Thus, at 402, the affinity of the conduit should be chosen to be the opposite of the fluid samples. Since most fluid samples contain water, it generally is desirable to choose hydrophobic materials for the conduits. Hence, glass and certain plastics may be chosen for the conduits. Other materials for the conduits may be chosen as well. However, if the fluid sample is hydrophobic, the conduits should be hydrophilic.

Another factor that may affect whether a fluid sample fragments in a pneumatic fluid transfer system is the inner diameter of the conduit that is chosen for use. Hence, at 404, an appropriate inner diameter for the conduits should be chosen. In general, smaller inner diameter tubes, such as 0.03 inch, 0.020 inch and 0.03 inch tubes can be difficult and pose a fragmentation risk. Conduits with diameters below 0.04 inch inner diameters or conduits with inner diameters larger than about 0.125 inch generally pose problems and should be avoided. In contrast, conduits inside the range extending between those limits, such as 1/16 inch tubes, work well.

Another parameter of interest is to determine the fluid packet size and the number of packets to be sent. At 406, the fluid packet size and the number of packets to be transported is determined. This effects how the source of the fluid (e.g., a bioreactor) is sampled. For purposes of this discussion, packets may be equated with samples or portions thereof, and a packet is a section of fluid contained in a conduit, like a tube. In general, the transport works better with small packet sizes rather than large packet sizes. It has been found that packets in the range of 50 μL to 1 mL are well suited for the pneumatic fluid transport systems described herein. That said, packet sizes of up to 5 mL should work well. The number of packets transported in a tube together should be limited as greater suction force or positive air pressure is required to move a larger number of packets relative to transporting a smaller number of packets. The number of packets of fluid transported together in a tube ideally is one packet but up to twenty packets may be transported together in the tube without incurring substantial problems. However, as packet sizes increase, the reliability of the system decreases, so the number of packets should be minimized.

At 408, the pump settings that move fluid samples at a suitable speed is chosen. The pump setting may vary based upon multiple factors, including but not limited to the composition of the fluid samples, the inner diameter of the conduits, the angle of orientation of the conduits relative to the floor, and the like. In general, fluid samples sent as packets need to self-adhere to move smoothly through a conduit without non-negligible fragmentation. If the packets do not self-adhere, the packets fragment into droplets that are prone to sticking to the inner surfaces of the conduits. When the positive air pressure or suction force on such fluid packets is too great, fluid blows through the fluid packets before the fluid packet can displace further down the transfer tube, and disperses over the inner walls of the conduits. Moreover, too great of positive air pressure or suction force may cause evaporation of the fluid packets. It has been found that the fluid packets need to be transported to their destination in a reasonable time frame, such as at most 10 or 20 minutes. The fluid samples, need to be treated within a time frame and need to be maintained at certain temperatures in many instances.

It has been found that a flow rate between 1 mL/min to 6 mL/min works well with a conduit that is a 1/16 inch inner diameter tube. That said, other flow rates may work well with different fluid sample compositions and/or different conduit inner diameters. A flow rate of 1.5 mL/min in a 1/16 inch tube has proven to move at a sufficient rate with an acceptable amount of fragmentation. Such settings resulted in successful transfer of fluid samples a distance in excess of 50 feet and a mass recovery of greater than 98% of the fluid samples in some instances. More generally, the mass recovery rate should be greater than 50%. The distance transported may be greater than 1 foot, 10 feet, 20 feet, 40 feet or 50 feet in some exemplary embodiments.

At 410, the pumps may be set at the determined settings. At 412, the system is operated as constructed and configured to transport the fluid samples.

It will be appreciated that various changes and form and detail may be made to the exemplary embodiments described herein without departing from the intended scope of the claims appended hereto and equivalents thereof.

1. A pneumatic fluid transport system, comprising:
a conduit for carrying a fluid from a source to a destination, wherein the conduit is at least 1 foot in length;
one or more pumps or vacuum sources secured to the conduit for applying a force to a gas for transporting the fluid along the length of the conduit; and
wherein the one or more pumps or vacuum sources are configured to move the fluid at a rate such that at least 50% of mass of the fluid is recovered at the destination.

2. The pneumatic fluid transport system of claim 1, wherein the conduit comprises one or more tubes.

3. The pneumatic fluid transport system of claim 2, wherein one end of the conduit is secured to a bioreactor from which the fluid is extracted.

4. The pneumatic fluid transport system of claim 2, wherein another end of the conduit is secured to a destination for processing a sample of the fluid.

5. The pneumatic fluid transport system of claim 1, wherein the one or more pumps or vacuum sources move the fluid at a flow rate of between 1 mL/min and 6 mL/min while transporting the fluid along the length of the conduit.

6. The pneumatic fluid transport system of claim 1, wherein the conduit has an inner diameter in the range of 0.3125 inches to 0.1 inches.

7. The pneumatic fluid transport system of claim 1, wherein the one or more pumps are secured to the conduit for applying the force to the gas for transporting the fluid and wherein the one or more pumps include a peristaltic pump.

8. The pneumatic fluid transport system of claim 1, wherein the conduit is at least 40 feet in length.

9. The pneumatic fluid transport system of claim 1, wherein at least 50% of the mass of the fluid is recovered at the destination.

10. The pneumatic fluid transport system of claim 1, wherein the fluid is a volume of 10 μL to 5 mL.

11. A system, comprising:
a bioreactor for providing a sample in fluid form;
a conduit connected to the bioreactor for receiving the sample, wherein the conduit is at least 1 foot in length;
a first pump connected to the tube for creating a pressure to transport the sample in fluid form along the conduit, the first pump being configured to move the fluid at a flow rate of between 1 mL/min and 6 mL/min; and
a first valve having a first position for receiving the sample into a sample loop and a second position to dispensing the sample out of the sample loop to a fluid path leading to sample processing equipment.

12. The system of claim 11, wherein the first valve further comprises a port connected to a fluid path leading to a second pump and a second valve.

13. The system of claim 12, wherein the second valve has a position for connecting with a wash source so that wash solution may be drawn and passed by the second pump to waste.

14. The system of claim 12, wherein the second valve has a position for connecting with an air source so that air may be drawn and passed by the second pump through the sample loop.

15. The system of claim 11, wherein the conduit has an inner diameter in the range of 0.3125 inches to 0.1 inches.

16. A method, comprising:
extracting a liquid sample from a bioreactor; and
using a pump configured to apply a force to a conduit that contains the liquid sample having an initial mass to transport the liquid sample at least 1 foot along the conduit at a flow rate between 1 mL/min and 2 mL/min so as to recover at least 50% of the initial mass of the liquid sample after transporting the liquid sample at least 1 foot.

17. The method of claim 16, wherein the conduit has an inner diameter in the range of 0.3125 inches to 0.1 inches.

18. The method of claim 16, wherein the conduit is hydrophobic.

19. The method of claim 16, wherein the liquid sample is transported at least 40 feet.

20. The method of claim 16, wherein the fluid sample contain at most 20 packets of fluid.
